(19) United States
(12) Patent Application Publication
(10) Pub. No.: US 2015/0182444 A1
(43) Pub. Date: Jul. 2, 2015

(54) TRADITIONAL CHINESE MEDICINE COMPOSITION FOR ANTI-WRINKLE

(71) Applicant: Yuan Yuan, Beijing (CN)

(72) Inventor: Yuan Yuan, Beijing (CN)

(73) Assignee: SHANG CHEN MEI BANG (BEIJING) BIOTECHNOLOGY CO., LTD., BEIJING (CN)

(21) Appl. No.: 14/144,564

(22) Filed: Dec. 31, 2013

(51) Int. Cl. A61K 8/97; A61K 8/64; A61K 8/60; A61K 8/44; A61K 8/65; A61K 8/99

(52) U.S. Cl. CPC A61K 8/97; A61K 8/65; A61K 8/99; A61K 8/60; A61K 8/44; A61K 8/64; A61K 2800/5922; A61Q 19/08

(57) ABSTRACT

The invention provides a composition for anti-medicine wrinkle relief, characterized in that said composition comprises the following composition of raw materials in parts by weight: 10-15 parts of water, 5-6 parts of collagen, silk, hydrolyzed 3-5 parts of collagen, milk protein 2-6 copies, copies of the product of yeast fermentation filtrate 3-5, 2-3 parts sucrose laurate, 4-6 parts oil of roses, chrysanthemum extract thousand Days 2-4 parts 3-6 parts of Salvia flower extract, collagen amino acids 3-5 parts, 1-2 parts frankincense extract, yeast peptide 3-4, 2-3 parts carbomer, 2-4 parts of mother of pearl extract, Ganoderma lucidum extract 3-5 parts. The composition can quickly penetrate the cortex, the absorption of various active ingredients depth help rebuild the skin matrix, abundance of skin firmness, smooth wrinkles, and texture.

### CROSS-REFERENCE TO RELATED APPLICATIONS

This application claims the priority benefit of Chinese Patent Application Serial No. 201310703312.4, filed on Dec. 19, 2013; Chinese Patent Application Serial No. 201310685138.5, filed on Dec. 13, 2013; Chinese Patent Application Serial No. 201310687223.5, filed on Dec. 13, 2013; Chinese Patent Application Serial No. 201310685115.4, filed on Dec. 13, 2013; Chinese Patent Application Serial No. 201310684837.8, filed on Dec. 13, 2013; Chinese Patent Application Serial No. 201310685112.0, filed on Dec. 13, 2013; Chinese Patent Application Serial No. 201310686861.5, filed on Dec. 13, 2013; Chinese Patent Application Serial No. 201310687218.4, filed on Dec. 13, 2013; Chinese Patent Application Serial No. 201310686865.3, filed on Dec. 13, 2013; Chinese Patent Application Serial No. 201310687250.2, filed on Dec. 13, 2013; Chinese Patent Application Serial No. 201310684846.7, filed on Dec. 13, 2013; Chinese Patent Application Serial No. 201310685166.7, filed on Dec. 13, 2013; and Chinese Patent Application Serial No. 201310687246.6, filed on Dec. 16, 2013, the disclosure of all of which are hereby incorporated by reference in their entirety.

### FIELD OF THE INVENTION

The invention relates to a traditional Chinese medicine composition for anti-wrinkle, belongs to a traditional Chinese medicine composition technology field.

### BACKGROUND OF THE INVENTION

In recent years, with the improvement of social economic development and living standards, people Antisenility, Beauty Totale, eternal youth more and more attention. However, due to modern work and accelerate the rate of Life Festival, some people just over three years old that started showing aging face and body, such as the minimalist looking, dull raw spots, wrinkles and more haggard, though now revealed in the skin, but the reason it is the body wasting organs, meridian obstruction caused by lack of blood, blood stasis leaving the gas, blood, sperm, was no more than the wing in the head and face caused by the outer kinky though related, but the “righteousness, evil can not dry”, such as peace and quiet internal organs, blood smooth, the evils from going to carry on. Current clinical treatment mainly treated with medication to achieve the purpose of prevention and recovery, but died in the rule table, you can not solve the problem fundamentally. There are oral prescription treatment, reaching internal therapeutic effect.

### SUMMARY OF THE INVENTION

The invention can quickly penetrate skin, deeply absorbed various active ingredients to help rebuild the skin matrix, abundance skin feeling firm, smooth wrinkles, texture.

Techniques of the present invention are: the kind of traditional Chinese medicine composition for anti-wrinkle relief, wherein said composition comprises the following parts by weight ratio of raw material composition: 10-15 parts water, silk collagen 5-6 copies, hydrolyzed collagen 3-5 parts, 2-6 parts milk protein, yeast ferment filtrate 3-5 parts, sucrose laurate 2-3 parts, 4-6 parts oil of roses, chrysanthemum extract object 2 thousand days—4 parts, parts 3-6 salvia flower extract, collagen amino acids 3-5 parts, 1-2 parts frankincense extract, yeast peptide 3-4, 2-3 parts Carbomer, mother of pearl extract 2-4 parts, Ganoderma lucidum extract 3-5 parts.

Among them, the ingredients may be: water 10-11 parts, parts 5-6 silk collagen, hydrolyzed collagen 3-4, 2-3 parts of milk protein, the product of yeast fermentation filtrate 3-4, sucrose laurate 2-3 copies, rose oil 4-5 copies Spilanthes extract 2-3 parts, 3-4 salvia flower extract, collagen, amino acids 3-4, 1-2 parts frankincense extract, yeast polypeptide Class 3-4, 2-3 parts Carbomer, mother of pearl extract 2-3 parts, 3-4 lucidum extract.

Wherein each component may be: 13-15 parts water, 5-6 parts of silk collagen, hydrolyzed collagen parts 4-5, 5-6 parts of milk protein, yeast fermentation filtrate of 4-5 parts of sucrose laurate 2-3 copies, rose oil 5-6 copies Spilanthes extract 3-4, 5-6 copies salvia flower extract, collagen, amino acids 4-5 parts, 1-2 parts frankincense extract, yeast polypeptide Class 3-4, 2-3 parts Carbomer, mother of pearl extract 3-4, 4-5 parts of Ganoderma lucidum extract.

The components are extracted through conventional methods together with accessories made from cream milk.

Also provides the use of traditional Chinese medicine composition described above, the amount of each of its usage is the size of a half-pearl face down about evenly extend onto the skin.

Pharmacological Effects:

Hydrolyzed Collagen: Widely used in neutral milk drinks, acidic milk drinks, fresh milk, yogurt and other liquid dairy towels, play against whey separation, emulsion stability and other effects, can also be added to milk, the milk can improve nutrition value, but also enhance the functionality of milk, accelerated bone development, enhance intelligence, improve the body's immune system.

Rose oil: Rose petals are distilled extract economic value is high. Can be used for medical, cosmetic, food, aromatherapy, research and other fields.

Spilanthes, alias Dengzhanhua, gerbera, gerbera, chrysanthemum Orangutans, sun flower, is a perennial herb, apical inflorescence, colors were red, white, yellow, orange, purple and so on.

Frankincense, whose real name is Lu Xun, alias Miss smoked incense, horsetail incense, incense nipple, the collapse of incense, incense Chak, Mollet incense, incense Duo Jialuo such as olive branch evergreen tree solidified resin.

Yeast: some single-celled fungus, is the history of human civilization is the earliest applications of microorganisms. Can survive in anoxic environments.

Carbomer: acrylic bonding allyl sucrose or pentaerythritol allyl ether polymer.

Mother of pearl: Powder shellfish nacre, with calming liver yang; sedative given shock; Liver eyesight.

Ganoderma lucidum: To basidiomycete Polyporaceae Ganoderma, or Ganoderma lucidum fruiting bodies grow in East Asia, is one of the best types of Ganoderma in efficacy.

After the composition of the present invention, the above ingredients, a synergistic effect of its efficacy.

### DETAILED DESCRIPTION OF EMBODIMENTS

### EXAMPLE 1

A medicine for anti-wrinkle composition of the buffer, wherein said composition comprises the following composition of raw materials in parts by weight: 10-11 parts of water, 5-6 parts of collagen, silk, hydrolyzed collagen 3-4, 2-3 parts of milk protein, yeast ferment filtrate 3-4, 2-3 parts sucrose laurate, 4-5 parts rose oil, a thousand days 2-3 parts chrysanthemum extract, salvia 3-4 flower extract, collagen amino acids 3-4, 1-2 parts frankincense extract, yeast peptide 3-4, 2-3 parts carbomer, mother of pearl extract 2-3 parts, Ganoderma lucidum extract 3-4.

### EXAMPLE 2

A medicine for anti-wrinkle composition of the buffer, wherein said composition comprises the following composition of raw materials in parts by weight: 13-15 parts of water, 5-6 parts of collagen, silk, hydrolyzed collagen 4-5 parts, 5-6 parts milk protein, yeast ferment filtrate 4-5 parts, 2-3 parts of sucrose laurate, 5-6 parts oil of roses, chrysanthemum extract thousand Days 3-4, Salvia 5-6 copies flower extract, collagen amino acids 4-5 parts, 1-2 parts of frankincense extract, yeast peptide 3-4, 2-3 parts carbomer, mother of pearl extract 3-4, Ganoderma lucidum extract 4-5 parts.

Usage: the amount of each one down about half the size of pearl face evenly extend onto the skin.

Clinical test: Test number 20, all adults.

Through the above human clinical trials, according to the daily morning and evening milk cream composition of the present invention is made onto the skin, one month after the statistical results: the effective rate was 100%. The composition of the extracellular matrix can help rebuild the epidermis, the skin abundance firm, gentle wrinkles, texture.

The following referenced cases are herein incorporated: U.S. Nonprovisional patent application Ser. No. 14/xxx,xxx, filed Dec. 30, 2013 with Attorney Docket No. ShangChen-13-01, and entitled “A traditional Chinese medicine composition that has the effect of soothing and cleaning face”; U.S. Nonprovisional patent application Ser. No. 14/xxx,xxx, filed Dec. 30, 2013 with Attorney Docket No. ShangChen-13-02, and entitled “A Traditional Chinese medicine extract composition with whitening skin care function and its preparation method”; U.S. Nonprovisional patent application Ser. No. 14/xxx,xxx, filed Dec. 30, 2013 with Attorney Docket No. Shangchen-13-03, and entitled “A traditional Chinese medicine composition for pigment repair”; U.S. Nonprovisional patent application Ser. No. 14/xxx,xxx, filed Dec. 30, 2013 with Attorney Docket No. Shangchen-13-04, and entitled “A traditional Chinese medicine composition for repair of acne”; U.S. Nonprovisional patent application Ser. No. 14/xxx,xxx, filed Dec. 30, 2013 with Attorney Docket No. Shangchen-13-06, and entitled “A traditional Chinese medicine composition for dispelling wrinkles and black eye and its preparation method”; U.S. Nonprovisional patent application Ser. No. 14/xxx,xxx, filed Dec. 30, 2013 with Attorney Docket No. Shangchen-13-07, and entitled “A Chinese medicine composition for nourishing body care and its preparation method”; U.S. Nonprovisional patent application Ser. No. 14/xxx,xxx, filed Dec. 30, 2013 with Attorney Docket No. Shangchen-13-08, and entitled “Chinese medicine composition that having acne cleansing effect”; U.S. Nonprovisional patent application Ser. No. 14/xxx,xxx, filed Dec. 30, 2013 with Attorney Docket No. Shangchen-13-09, and entitled “An anti-aging skin rejuvenation medicine composition and its preparation method”; U.S. Nonprovisional patent application Ser. No. 14/xxx,xxx, filed Dec. 30, 2013 with Attorney Docket No. Shangchen-13-10, and entitled “Chinese medicine composition for pigment desalination”; U.S. Nonprovisional patent application Ser. No. 14/xxx,xxx, filed Dec. 30, 2013 with Attorney Docket No. Shangchen-13-011, and entitled “Chinese medicine composition for acne scar repair”; U.S. Nonprovisional patent application Ser. No. 14/xxx,xxx, filed Dec. 30, 2013 with Attorney Docket No. Shangchen-13-012, and entitled “Chinese medicine composition for Prevent from aging and soothing the skin”; and U.S. Nonprovisional patent application Ser. No. 14/xxx,xxx, filed Dec. 30, 2013 with Attorney Docket No. Shangchen-13-013, and entitled “Chinese medicine composition for Nourishing the skin and its preparation method”, the disclosure of all of which are herein incorporated by reference in its entirety.

What is claimed is:

1. A Chinese medicine for anti-wrinkle relief composition, wherein said composition comprises the following parts by weight ratio of raw material composition: 10-15 parts water, 5-6 parts silk collagen, hydrolyzed collagen 3 -5 copies 2-6 copies milk protein, yeast ferment filtrate 3-5 parts, sucrose laurate 2-3 parts, 4-6 parts rose oil, 2-4 '000 days purpurea extract, salvia flower 3-6 copies of extracts, collagen amino acids 3-5 parts, 1-2 parts frankincense extract, yeast peptide 3-4, 2-3 parts Carbomer, mother of pearl extract 2-4 copies, Ganoderma lucidum Extract 3-5 parts.

2. Chinese medicine composition as claimed in claim 1, wherein, wherein the components are: 10-11 parts water, 5-6 parts of silk collagen, hydrolyzed collagen 3-4, 2-3 parts milk protein, yeast fermentation product filtrate 3-4, 2-3 parts sucrose laurate, 4-5 parts rose oil, a thousand days 2-3 parts chrysanthemum extract, salvia flower extract 3-4, collagen amino acids 3-4, 1-2 parts frankincense extract, yeast peptide 3-4, 2-3 parts Carbomer, mother of pearl extract 2-3 parts, 3-4 lucidum extract.

3. Chinese medicine composition as claimed in claim 1, wherein, wherein the components are: 13-15 parts water, collagen, silk parts 5-6, 4-5 parts of hydrolyzed collagen, 5-6 parts of milk protein, yeast fermentation product filtrate 4-5 parts, 2-3 parts of sucrose laurate, 5-6 parts oil of roses, chrysanthemum extract Thousand Days 3-4, 5-6 copies salvia flower extract, collagen, amino acids 4-5 parts, 1-2 parts frankincense extract, yeast peptide 3-4, 2-3 parts Carbomer, mother of pearl extract 3-4, 4-5 parts of Ganoderma lucidum extract.

4. Chinese medicine composition as claimed in claim 1, wherein the components extracted through conventional methods together with accessories made from cream milk.

5. The Essence of Chinese medicine made composition according to claim its use as a pearl-sized amount for each half face down about a uniform extension onto the skin.
